(19) United States
(12) Patent Application Publication
(10) Pub. No.: US 2012/0265306 A1
(43) Pub. Date: Oct. 18, 2012

(54) SPINAL IMPLANT WITH ATTACHABLE BONE SECURING COMPONET

(75) Inventor: Hai H. Trieu, Cordova, TN (US)

(73) Assignee: Warsaw Orthopedic, Inc., Warsaw, IN (US)

(21) Appl. No.: 13/086,994

(22) Filed: Apr. 14, 2011

(51) Int. Cl. A61F 2/44

(52) U.S. Cl. 623/17.16

(57) ABSTRACT

A spinal implant for insertion into an intervertebral disc space for intervertebral stabilization, the implant comprising a radiopaque substrate having bone securing serrations coupled to a radiolucent insert. The implant's radiolucent and radiopaque properties facilitate radiographic assessment of fusion across the disc space, assessment of osseointegration between vertebral endplates and osseointegration of the implant to adjacent vertebral end plates. The implant comprises an implant substrate having at least one insert cavity and bone securing serrations, and at least one insert component, whereby the insert component is configured to be securely coupled to the implant substrate via the at least one insert cavity thereby forming the implant. The implant preferably comprises a Titanium (Ti) substrate coupled to a polyetheretherketone (PEEK) insert component whereby the implant serrations are positioned between adjacent vertebral endplates when the implant is inserted into the disc space thereby securely positioning the implant between the adjacent vertebrae.

### BACKGROUND

The present application is directed to implants, devices and methods for stabilizing vertebral members, and more particularly, to intervertebral implants, devices and methods of use in replacing, in whole or in part, an intervertebral disc, a vertebral member, or a combination of both to distract and/or stabilize the spine.

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve. Intervertebral discs are positioned between the vertebral members and permit flexion, extension, lateral bending, and rotation.

Various conditions and ailments may lead to damage of the spine, intervertebral discs and/or the vertebral members. The damage may result from a variety of causes including, but not limited to, events such as trauma, a degenerative condition, a tumor, or infection. Damage to the intervertebral discs and vertebral members can lead to pain, neurological deficit, and/or loss of motion of the spinal elements.

Various procedures include replacing a section of or an entire intervertebral disc, a section of or an entire vertebral member, or both. One or more spinal implants may be inserted to replace damaged discs and/or vertebral members. The implants are configured to be inserted into an intervertebral space and contact against adjacent vertebral members. The implants are intended to reduce or eliminate the pain and neurological deficit, and increase the range of motion.

The curvature of the spine and general shapes of the vertebral members may make it difficult for the implants to adequately contact the adjacent vertebral members or to position the adjacent vertebral members in a desired orientation. There is a need for spinal implants or devices configurable to match the spinal anatomy for secure contact and/or desired orientation of the spinal implants or devices implanted into an intervertebral disc space.

### SUMMARY

The present application discloses a spinal implant for insertion into and positioning in an intervertebral disc space. The implant comprises an implant substrate comprising at least one insert cavity and bone securing serrations, and at least one insert component. The at least one insert component and is configured to be securely coupled to the implant substrate via entry of the at least one insert component into the at least one insert cavity via a lateral sidewall thereby forming the spinal implant. The insert component is internally positioned inside the implant substrate when securely coupled to the implant substrate. The at least one insert cavity and the at least one insert component are securely coupled via a mechanical interlock configuration. In an alternative aspect, the spinal implant comprises one insert component configured to be securely coupled to the implant substrate via a first or second insert cavity to thereby form the spinal implant. In such an embodiment, the one insert component, when securely coupled to the implant substrate, laterally spans across the implant substrate between a first and second lateral sidewall. In a preferred aspect, the implant substrate is comprised of a radiopaque titanium (Ti) or metallic material and the at least one insert component is a polyetheretherketone (PEEK) or resorbable material. Additionally, the implant substrate may be coated with a Hydroxyapatite (HA) layer.

The present application also discloses a spinal implant for insertion into and positioning in an intervertebral disc space. The implant comprises an implant substrate comprising at least one insert cavity and bone securing serrations, and at least one insert component configured to be positioned inside the at least one insert cavity. The at least one insert component is configured to be securely coupled to the implant substrate via a lateral sidewall entry into the at least one insert cavity to thereby form the spinal implant. The at least one insert cavity and the at least one insert component are securely coupled via a mechanical interlock configuration. In another aspect, the spinal implant comprises one insert component configured to be securely coupled to the implant substrate via a first or second insert cavity to thereby form the spinal implant. In such an embodiment, the one insert component, when securely coupled to the implant substrate, laterally spans across the implant substrate between a first and second lateral sidewall. In a preferred aspect, the implant substrate is comprised of a radiopaque titanium (Ti) or metallic material and the at least one insert component is a polyetheretherketone (PEEK) or resorbable material. Additionally, the implant substrate may be coated with a Hydroxyapatite (HA) layer.

There is further provided a spinal implant for insertion into an intervertebral disc space for intervertebral stabilization, the implant comprising a radiolucent polymer substrate coupled to a radiopaque and osseoconductive bone securing component

There is further provided a spinal implant for insertion into an intervertebral disc space for intervertebral stabilization. The implant comprises a radiopaque implant substrate having bone securing serrations coupled to a radiolucent insert which provides the spinal implant with secure fixation within the intervertebral disc space and adjacent vertebrae. The disclosed spinal implant includes radiolucent, radiopaque and osseointegrative properties that facilitate radiographic assessment of fusion across the disc space, assessment of osseointegration between vertebral endplates and osseointegration of the spinal implant to adjacent vertebral end plates.

The present application also discloses a biocompatible spinal implant for insertion into an intervertebral space between adjacent vertebral members. The implant imparts, distracts and restores desired disc space height in adjacent vertebral bodies when the implant is positioned in the intervertebral disc space and enables fusion of the adjacent vertebrae. The implant comprises a radiopaque metallic implant substrate having bone securing serrations coupled to a radiolucent polyetheretherketone (PEEK) insert component which enable the spinal implant to be securely positioned in the intervertebral disc space between adjacent vertebral endplates. In a preferred aspect, the implant substrate is preferably a titanium (Ti) material or a titanium (Ti) alloy.

The various aspects of the various embodiments may be used alone or in any combination, as is desired. Disclosed aspects or embodiments are discussed and depicted in the attached drawings and the description provided below.

### BRIEF DESCRIPTION OF THE DRAWINGS

FIG. 1 is sagittal plane view of an implant according to one embodiment of the present disclosure positioned in an intervertebral space between vertebral members;

FIG. 2 is a perspective view of an implant according to one embodiment of the present disclosure;

FIG. 3 is a side view of the spinal implant of FIG. 2;

FIG. 4 is a top perspective view of the spinal implant FIG. 2;

FIG. 5A is a perspective view of the implant of FIG. 3 along section line A-A;

FIG. 5B is a perspective view of the implant of FIG. 4 along section line B-B;

FIG. 5C is a perspective view of the implant of FIG. 4 along section line C-C;

FIG. 6A is a perspective view of an implant according to a second embodiment of the present disclosure;

FIG. 6B is a perspective view of the implant of FIG. 6A along section line D-D;

FIG. 7A is a perspective view of an implant according to a third embodiment of the present disclosure;

FIG. 7B is a perspective view of the implant substrate of the implant of FIG. 7A; and

FIG. 7C is a perspective view of the implant insert of the implant of FIG. 7A.

### DETAILED DESCRIPTION

The present disclosure is directed to intervertebral implants for spacing apart vertebral members. The present disclosure relates to medical devices such as spinal intervertebral implants implanted between adjacent vertebral bodies of a spinal column section, and methods of use. More particularly, to a spinal implant with a metallic implant substrate coupled to a polymer insert component where the implant substrate includes surface serrations, teeth, texture or extensions which enable the spinal implant to be securely positioned between adjacent vertebral endplates. The implant imparts, distracts and restores desired disc space height in adjacent vertebral bodies when the implant is positioned in the intervertebral disc space. The disclosed spinal implant includes radiolucent, radiopaque and osseointegrative properties that facilitate radiographic assessment of fusion or the bridging bone mass across the disc space while reducing stress shielding effects, radiographic assessment of osseointegration between vertebral endplates and implant surfaces, and osseointegration of the spinal implant to adjacent vertebral bodies. For purposes of promoting an understanding of the principles of the invention, reference will now be made to one or more embodiments or aspects, examples, drawing illustrations, and specific language will be used to describe the same. It will nevertheless be understood that the various described embodiments or aspects are only exemplary in nature and no limitation of the scope of the invention is thereby intended. Any alterations and further modifications in the described embodiments or aspects, and any further applications of the principles of the invention as described herein are contemplated as would normally occur to one skilled in the art to which the invention relates.

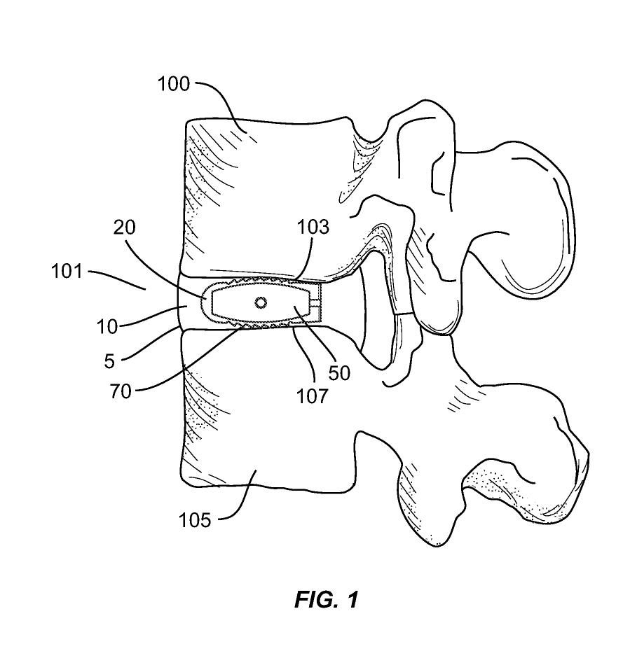

FIG. 1 illustrates a sagittal plane view of vertebral joint section or motion segment of a vertebral column. A spinal implant or device 10 is positioned in an intervertebral disc space 101 between adjacent vertebral members 100 and 105. The upper and lower vertebral bodies 100 and 105 include respective end plates 103 and 107. An intervertebral disc space 101 is located between the endplates 103 and 107. An intervertebral disc 5 is located in the intervertebral disc space 101 between the adjacent endplates 103 and 107 and around the periphery of the disc space 101. The intervertebral disc 5 is comprised of an annulus fibrosus or annulus which surrounds a nucleus pulposus. FIG. 1 further depicts a spinal implant, spacer or device 10, with attachable insert components 50 and 51, positioned in the intervertebral disc space 101. The spinal implant 10 can be used to promote fusion or preserve motion between adjacent vertebral bodies 100 and 105, depending on the specific shape or configuration of the implant used in a surgical procedure.

FIG. 1 depicts an implantation technique where the spinal implant 10 has been delivered to the intervertebral disc space 101, for example via a known surgical technique such as a posterior lumbar interbody fusion (PLIF) approach and procedure. Such a spinal implant PLIF procedure and approach is a well known surgical implant procedure and delivery approach for delivery and insertion of a spinal implant 10 into a desired or selected intervertebral disc space 101. Those of skill in the art will recognize that the spinal implant 10 could also be delivered and inserted in the disc space 101 so as to have different orientations and positions in the disc space 101 between the adjacent vertebrae 100 and 105. For example using known surgical approaches, including, anterior, posterior, direct lateral, translateral, posterolateral, anterolateral or any other suitable oblique direction desired or required by a surgeon or medical application. The spinal implant 10 could also be delivered and inserted in the disc space 101 using other well known surgical procedures and techniques, including among others, anterior lumbar interbody fusion (ALIF), direct lateral lumbar interbody fusion (DLIF), transforaminal lumbar interbody fusion (TLIF) or other known surgical procedures or techniques desired or required by a surgeon or medical application. Further, those of skill in the art will also recognize that a spinal implant 10 may be delivered and inserted through known surgical techniques and procedures via open, mini-open, minimal access spinal technologies (MAST) or other minimally invasive surgical (MIS) techniques. Moreover, delivery and insertion of the present spinal implant 10 is contemplated through the use of typical and existing instruments presently known and used in existing surgical approached, procedures and techniques.

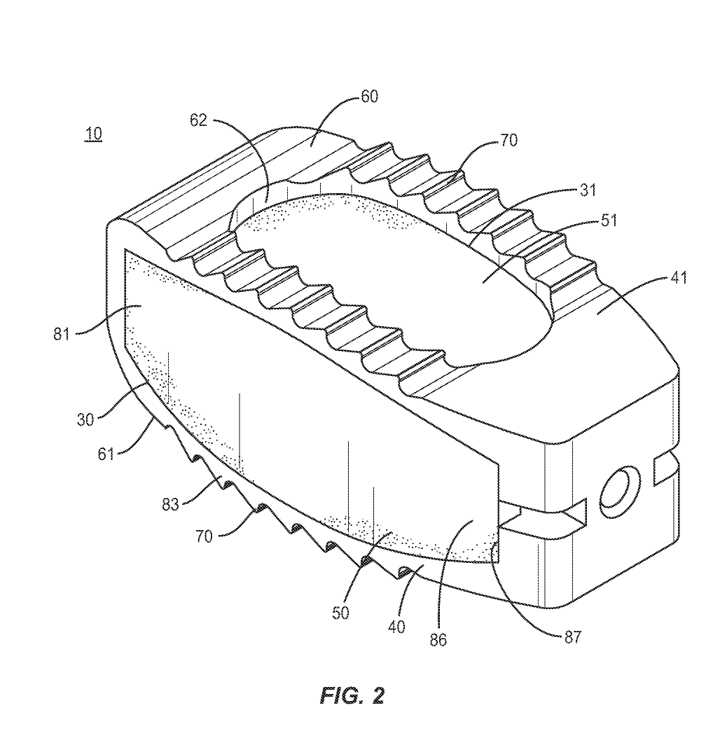

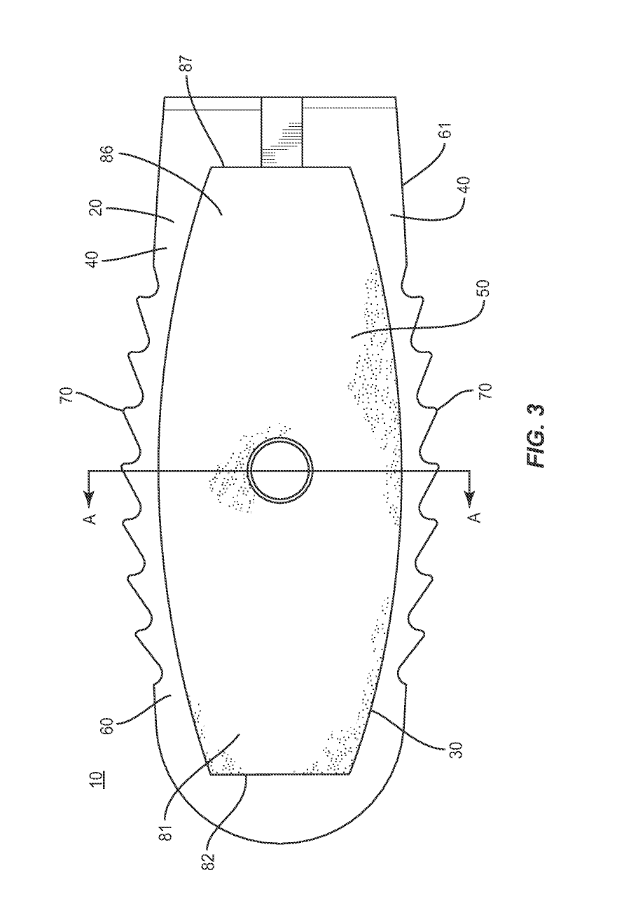

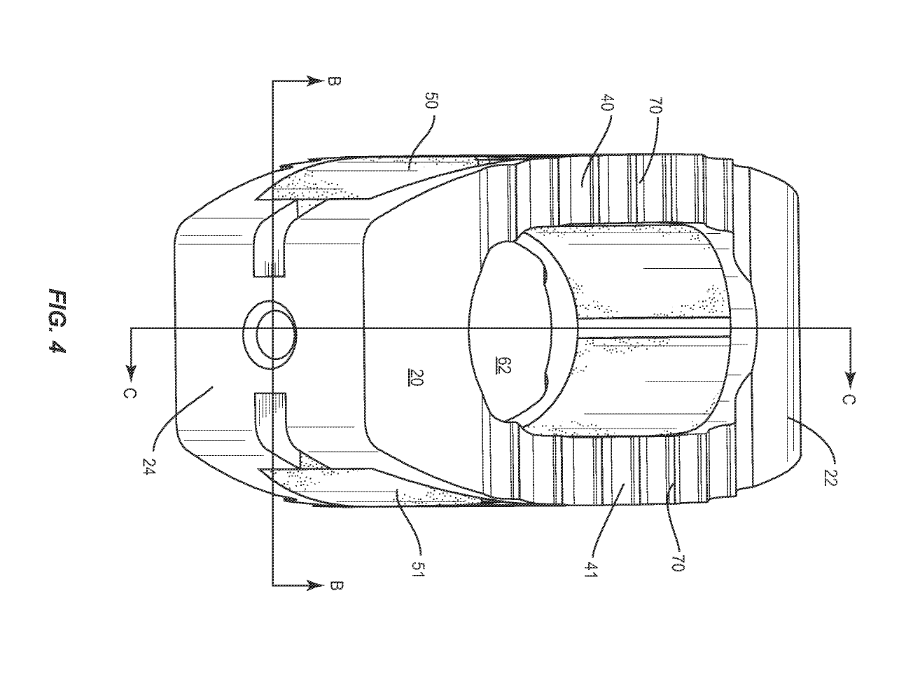

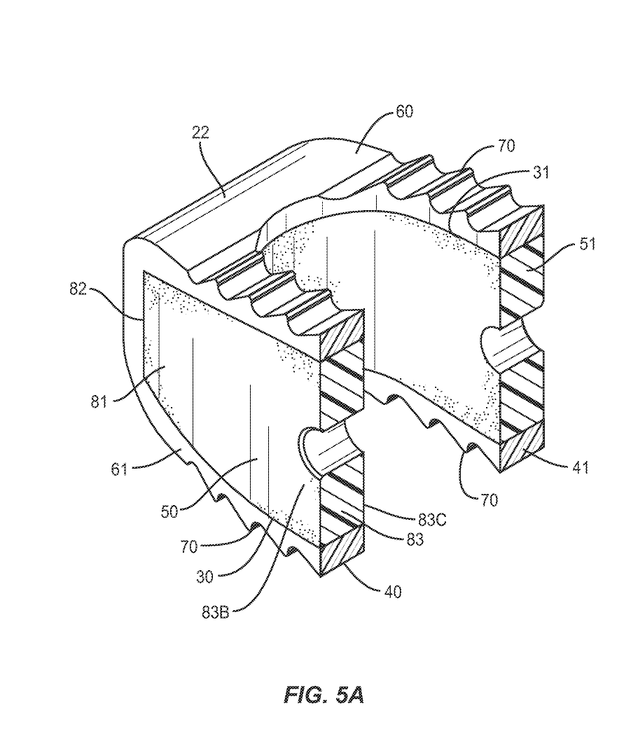

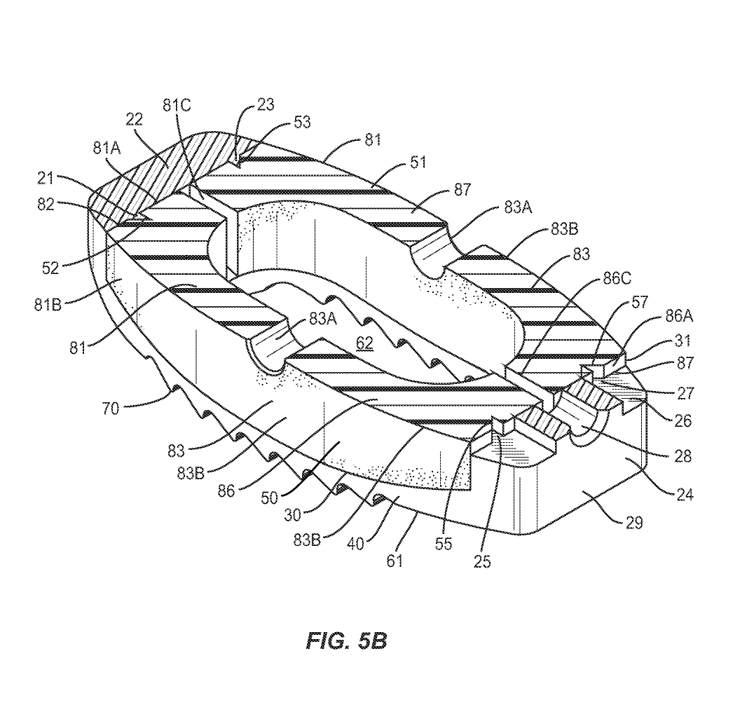

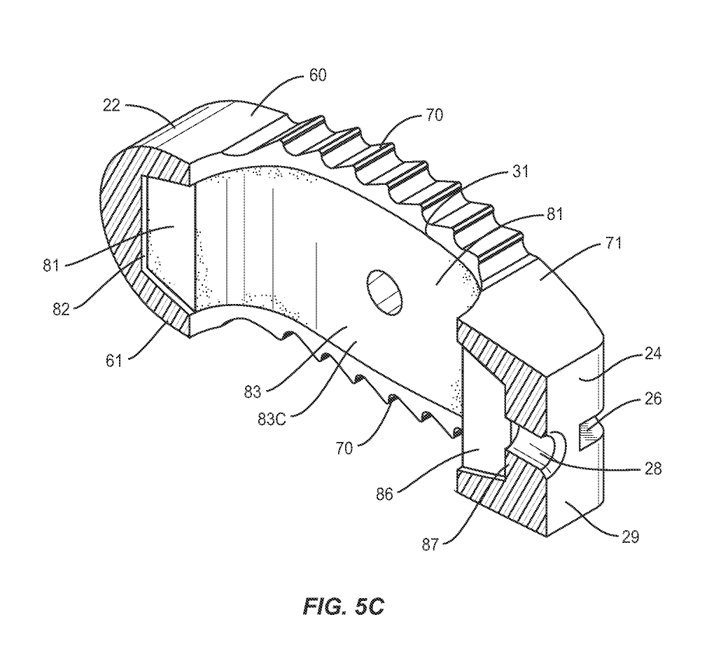

FIGS. 2-5C illustrate a spinal implant 10 according to a preferred aspect of the present disclosure. FIG. 2 is perspective view of the preferred spinal implant 10. FIG. 3 is a side view of the spinal implant 10 of FIG. 2. FIG. 4 is a top perspective view of the spinal implant 10 of FIG. 2. FIG. 5A is a perspective view of the spinal implant 10 of FIG. 3 along section line A-A. FIGS. 5B and 5C are perspective views of the spinal implant 10 of FIG. 4 along section lines B-B and C-C, respectively. The implant 10 comprises an implant body or substrate 20 and first and second insert components 50 and 51 which are laterally attached to the implant body 20. In the embodiment shown in FIG. 1, there is shown a first insert component 50 of the spinal implant 10 secured or attached to a first implant lateral section or sidewall 40, and a second insert component 51 of the spinal implant 10 secured or attached to a second implant lateral section or sidewall 41. In the preferred aspect of the spinal implant 10, the first and second insert components 50 and 51 are respectively laterally inserted and secured or attached to first and second lateral implant sections or sidewalls 40 and 41.

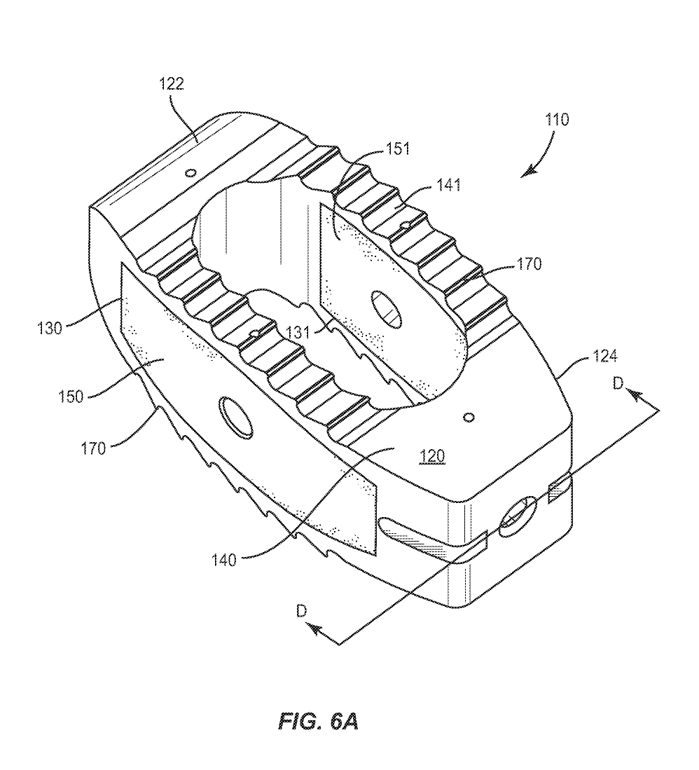

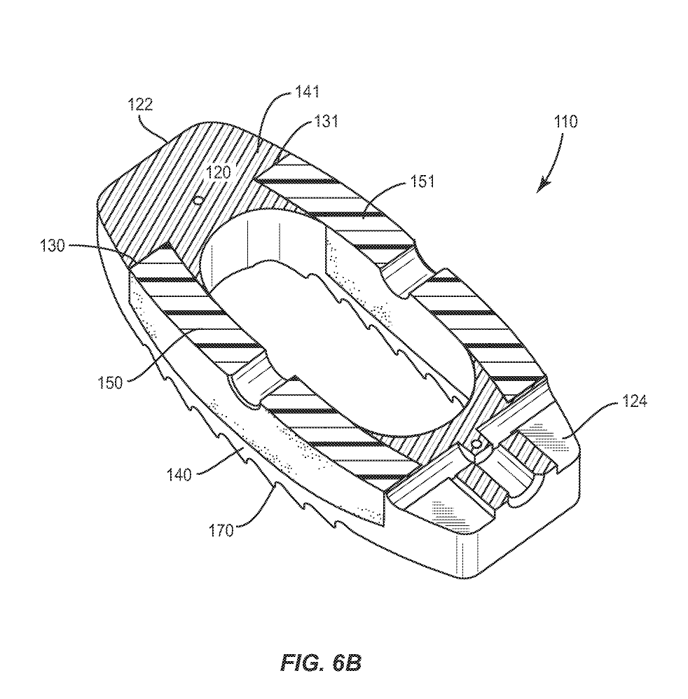

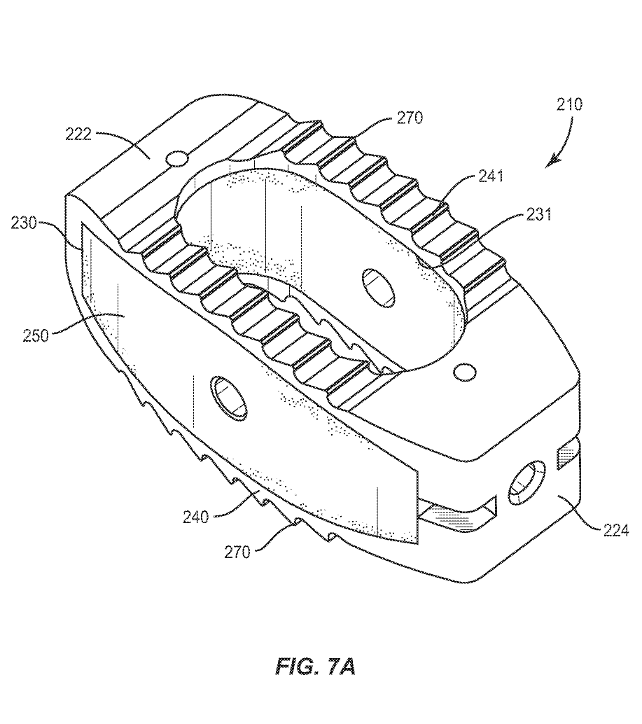

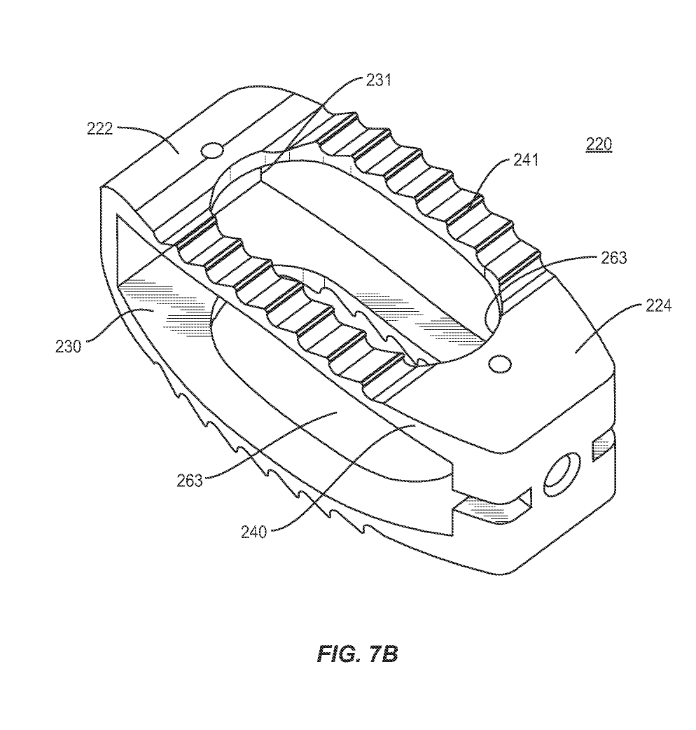

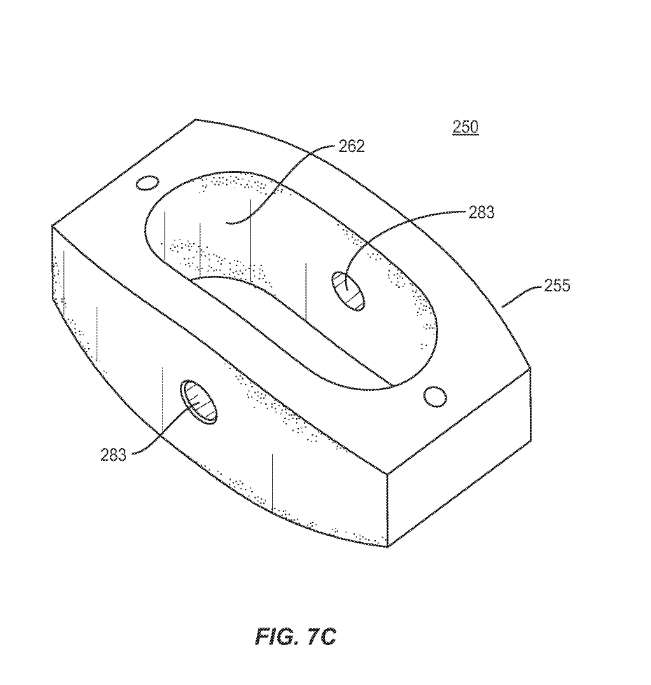

In the preferred aspect shown in FIGS. 2-5C, there are two identical insert components 50 and 51 which respectively insert into the insert cavities 30 and 31. However, those of skill in the art will recognize that the first and second insert components 50 and 51 may take on a different sizes and configurations. For example, FIGS. 6A-6B shows first and second insert components 150 and 151 that are smaller in size and have a different configuration compared to the first and second insert components 50 and 51 shown in FIGS. 2-5C. Additionally, those of skill in the art will recognize that that instead of two insert components 50 and 51, a spinal implant 10 may instead have a single insert component 250 that is inserted into the implant substrate 220 and secured or attached to the implant body or substrate 220, as shown in FIGS. 7A-7C. In such a case, the single insert component 250 would spans the implant substrate 220 from the first to second lateral implant section or sidewall 240 and 241, for example as show in FIGS. 7A-7C. Whether one or more insert components 50, 51, 150, 151 or 250 are used with the implant substrate 20, 120 and 220 will depend on the selection or requirements of a surgeon or medical procedure or application. Additionally, the spinal implant 10 can comprise a shape, configuration or size that may be needed by a surgeon or a spinal implant procedure or application. FIGS. 2-7C show different aspects of the spinal implant with attachable insert or inserts.

In a preferred aspect, the spinal implant 10 comprises a substrate 20 and a pair of insert components 50 and 51. The substrate 20 comprises a leading end 22 which has a substantially curved or rounded surface to permit the implant body 10 to distract collapsed or semi-collapsed adjacent vertebral bodies 100 and 105 when the implant 10 is introduced or inserted into a disc space 101. The implant 10 also includes a rear end 24 with a recess section 26 and orifice 28 extending inwardly in a direction from a rear end wall 29 toward the implant's leading end 22. The recess section 26 and orifice 28 provide a means to attach an instrument (not show) to grasp, attach to and manipulate the insertion and orientation of the spinal implant 10 as the implant 10 is delivered to a selected or desired disc space 101.

The spinal implant substrate 20 further comprises first and second lateral sections or sidewalls 40 and 41 between the leading end 22 and rear end 24. In the preferred embodiment, there is a first insert cavity 30 substantially centered in the first lateral sidewall 40 configured to accept a complimentarily configured insert component 50, and partially extends into the implant's leading and rear ends 22 and 24. The first insert cavity 30 is defined and positioned between the upper and lower portions of the first lateral sidewall 40 and the substrate's leading end 22 and rear end 24. Additionally, there is a corresponding or opposing second insert cavity 31 substantially centered in the second lateral sidewall 41 and partially extends into the implant's leading and rear ends 22 and 24. The second insert cavity is also configured to accept a complimentarily configured insert component 51. The second insert cavity 31 is defined and positioned between the upper and lower portions of the second lateral sidewall 41 and the substrate's leading end 22 and rear end 24. Those of skill in the art will readily recognize that the insert cavity 30 or 31 may have a configuration where the insert cavity 30 or 31 only minimally or does not extend into the implant's leading and rear ends 22 and 24 or only minimally extends into the into the implant's leading and rear ends 22 and 24 such that the insert cavity 30 or 31 is substantially or entirely positioned in a lateral sidewall 40 or 41. For example as shown in FIG. 6A-6B, where the insert cavities 130 and 131 are substantially positioned in a lateral sidewall 40 or 41. The insert cavity 30 or 31 configuration and position on the implant substrate 20 will depend on the selection or requirements of a surgeon or medical procedure or application.

The insert cavities 30 and 31 will facilitate the placement of the insert components 50 and 51 into the implant substrate 20 by permitting sliding entry of the insert components 50 and 51 therein. The insert cavities 30 and 31 and the insert components 50 and 51 preferably have complimentary and corresponding configurations such that the implant substrate 20 and insert components 50 and 51 can be lockingly engaged when the insert components 50 and 51 are slideably coupled or combined with the implant substrate 20. Those of skill in the art will recognize that the insert component 50 and 51 and corresponding insert cavities 30 and 31 can have a variety of complimentary and cooperating configurations which permit the insertion of the insert components 50 and 51 into the implant substrate 20. The locking or secure engagement between the insert components 50 and 51 and the implant substrate insert cavities 30 and 31 can be a result from the complimentary and corresponding configurations of the insert components 50 and 51 and insert cavities 30 and 31. This mechanical coupling or locking engagement is or can be a friction fit or interference fit. Those of skill in the art will recognize that other locking or engagement mechanisms may be use to securely couple the insert components 50 and 51 to the implant substrate 20, for example a rough or uneven surface interface between the insert components 50 and 51 and insert cavities 30 and 31 to increase the friction fit strength. Other attachment means contemplated to securely couple the insert components 50 and 51 to the implant substrate 20 include pins, rivets, screws, bolts and nuts, adhesive bonding, thermal bonding, dove tail, mechanical interlocking, over-molding, insert molding, or combinations thereof.

In the preferred embodiment, the spinal implant substrate 20 further comprises insert securing tenons 21, 23, 25 and 27 which cooperate with corresponding insert component channels 52, 53, 55 and 57 to augment the locking or engagement between the insert components 50 and 51 to the implant substrate 20, when the insert components 50 and 51 are inserted into the insert cavities 30 and 31. The first and second insert securing tenons or extensions 21 and 23 extend inwardly into the insert cavities 30 and 31 away from the interior of the substrate's leading end 22, best shown in FIG. 5B. The spinal implant substrate 20 also comprises opposing insert securing tenons or extensions 25 and 27 which extend inwardly into the insert cavities 30 and 31 from the interior of the substrate's rear end 22. In this embodiment, the insert securing tenons 21, 23, 25 and 27 are configured to be in a substantially vertical orientation and have a triangle or pyramid type shape. The insert securing tenons 21, 23, 25 and 27 are configured to engage corresponding insert component channels 52, 53, 55 and 57 when the insert components 50 and 51 are coupled or combined with the implant substrate 20 and brought into locking engagement with each other. The insert securing tenons 21, 23, 25 and 27 in combination with the insert component channels 52, 53, 55 and 57 facilitate the placement of the insert components 50 and 51 in a final locking position in the implant substrate 20 through the mechanical interlock of the insert components 50 and 51 and the implant substrate 20. The insert component channels 52, 53, 55 and 57 will preferably have a configuration that correspond to the substantially vertical orientation and triangle or pyramid type shape insert securing tenons 21, 23, 25 and 27. In this case, the insert component channels 52, 53, 55 and 57 will have a substantially vertical orientation and a channel with a triangle or pyramid type cross-section. Those of skill in the art will recognize that more or less corresponding insert securing tenons 21, 23, 25 and 27 and insert component channels 52, 53, 55 and 57 may be used to securely couple the insert components 50 and 51 to the implant substrate 20. In the preferred aspect, shown in FIG. 3, there are four insert securing tenons 21, 23, 25 and 27 and four corresponding insert component channels 52, 53, 55 and 57.

As the insert components 50 and 51 are inserted into the insert cavities 30 and 31, the securing tenons 21, 23, 25 and 27 and insert component channels 52, 53, 55 and 57 travel towards each other until the securing tenons 21, 23, 25 and 27 are positioned inside the insert component channels 52, 53, 55 and 57. At this point, the insert components 50 and 51 are securely coupled to the implant substrate 20. The combined securing tenons 21, 23, 25 and 27 and insert component channels 52, 53, 55 and 57, which are now securely engaged and mechanically interlocked, augment or supplement the friction fit or interference fit between the implant substrate 20 and insert components 50 and 51. This augmented mechanical coupling or locking engagement comprises both a friction fit or interference fit and mechanical interlock between the implant substrate 20 and insert components 50 and 51. Those of skill in the art will recognize that the insert securing tenons 21, 23, 25 and 27 and insert component channels 52, 53, 55 and 57 can have a variety of complimentary configuration which can secure and lockingly engage the insert components 50 and 51 to the implant substrate 20.

In the preferred aspect shown in FIGS. 2-5C, there are two identical insert components 50 and 51 which respectively insert into the insert cavities 30 and 31. Those of skill in the art will readily recognize that non-identical insert components 50 and 51 may instead be used as may be selected or required by a physician, procedure or medical application. In a case where insert components 50 and 51 with non-identical size or configuration are used, corresponding insert cavities 30 and 31 will have complimentary size and configuration to match that of the insert components 50 and 51 that will be positioned therein. Further, a single insert component 250 may also be used to span the insert cavities 230 and 231 between the first and second lateral side walls 240 and 241, as shown in FIGS. 7A-7C. In such a case as shown in FIGS. 7A-7C, the insert cavities 230 and 231 will be configured and sized to accept the single insert component 250,

The spinal implant substrate 20 also comprises an upper implant surface 60 defined by the upper portions of the leading end 22, rear end 24, first lateral sidewall 40 and second lateral sidewall 41, and a lower implant surface 61 defined by the lower portions of the leading end 22, rear end 24, first lateral sidewall 40 and second lateral sidewall 41. The upper implant surface 60 comprises an implant substrate aperture 62 defined by the upper portions or sections of the substrate's leading end 22, rear end 24 and first and second lateral sidewalls 40 and 41. The lower implant surface 61 comprises an opposing implant substrate aperture 62 defined by the lower portions or sections of the substrate's leading end 22, rear end 24 and first and second lateral sidewalls 40 and 41. The implant's 10 substrate aperture 62 is configured such that that there is a substantially vertical channel or cavity that extends between and through the upper and lower implant surfaces 60 and 61. Additionally, the substantially vertical implant channel 62, in conjunction with the first and second insert cavities 30 and 31, define a substantially lateral or horizontal cavity that extends laterally across and through the implant substrate 20 between the implant's first and second later sidewalls 40 and 41. This will permit insertion of one or more insert components 50, 51, 150, 151 or 250 into the implant substrate 20, 120 or 220.

In a preferred embodiment, the implant substrate apertures 62 permit the insertion of a graft material which assists in promoting fusion 100 and 105 of the adjacent vertebrae at the disc space 101 where the implant 10 is inserted. The graft material may be composed of any type of material that has the ability to promote, enhance and/or accelerate the bone growth and fusion or joining together of the vertebral bodies 100 and 105 by one or more fusion mechanisms such as osteogenesis, osteoconduction and/or osteoinduction. The graft material may include allograft material, bone graft, bone marrow, a demineralized bone matrix putty or gel and/or any combination thereof. The graft filler material may promote bone growth through and around the substrate aperture 62 to promote fusion of the intervertebral joint 100 and 105. Those of skill in the art will recognize that the use of filler graft material is optional, and it may or may not be used depending on the needs or requirements of a physician or a medical procedure.

The implant substrate 20 further comprises upper and lower bone securing surface serrations, teeth, projections or extensions 70 which enable the spinal implant 10 to be securely positioned between adjacent vertebral endplates and act as anti-ejection aspects for the coupled implant 10. The surface serrations 70 preferably extend outwardly from the upper and lower implant surfaces 60 and 61. In the upper implant surface 60, the surface serrations 70 are preferably configured and positioned on the upper portions or sections of the first and second lateral sidewalls 40 and 41, between the substrate's leading end 22 and rear end 24. And, in the lower implant surface 61, the surface serrations 70 are preferably configured and positioned on the opposing lower portions or sections of the first and second lateral sidewalls 40 and 41, between the substrate's leading end 22 and rear end 24.

The implant serrations 70 directly interact with and engage the vertebral endplates 103 and 109 when the spinal implant 10 is positioned in the disc space 101 and provide, in part, stability of the implant 10 in the disc space 101 between adjacent vertebrae 100 and 105. In the preferred embodiment, the implant serrations or teeth 70 are preferably oriented in a rear lean direction such that the teeth or serrations 70 are oriented away or opposite the implant leading end 22 and toward the implant rear end 24. In this manner, the rear leaning orientation of the teeth or serrations 70 provide minimal resistance when the spinal implant is being inserted into a disc space 101. Once inserted, the rear leaning orientation of the teeth or serrations 70 provide a mechanism to prevent the assembled spinal implant 10 from being ejected, or minimize or retard implant movement in a direction tending to eject the implant 10 from the disc space 101, once the spinal implant 10 is positioned in the disc space 101. In the aspects shown in FIGS. 2-7C, the teeth or serrations 70 are generally triangular in shape when viewed from a side profile. Those of skill in the art will recognize that the teeth or serrations 70 can instead have other shapes, configurations and sizes including, among others, pyramids, triangles, cones, spikes and keels, as well as different teeth or serration orientation, as may be needed or desired by a physician, procedure or medical application.

In the preferred aspect shown in FIGS. 2-5C, there are two identical inserts or insert components 50 and 51 which respectively insert into and attach to corresponding substrate insert cavities 30 and 31 to lockingly engage or mechanically couple the insert components 50 and 51 to the implant substrate 20. Those of skill in the art will readily recognize that non-identical insert components 50 and 51 can also be used as may be selected or required by a physician, procedure or medical application so long as the inserts 50 and 51 can lockingly engage or mechanically couple the complimentary and corresponding insert cavities 30 and 31 in the implant substrate 20. Further, a single insert component 250 may also be used, as shown in FIG. 7A, as may be selected or required by a physician, procedure or medical application. The insert component 50 or 51 and corresponding implant substrate cavity 30 or 31 will have complimentary configurations so as to permit them to be combined or coupled in a locked or engaged position, as shown in FIGS. 2 and 5C.

In the preferred aspect shown in FIGS. 2-5C, an insert component 50 or 51 comprises a leading insert end 81, a rear insert end 86 and a central insert section 83 between the leading insert end 81 and rear insert end 86. When insert component 50 or 51 is coupled, locked or engaged to the implant substrate 20, the insert component 50 or 51 will be positioned in corresponding implant insert cavities 30 and 31. In the coupled position of the preferred aspect, the leading insert end 81 is adjacent the interior facing surface 82 of the substrate's leading end 22. The rear insert end 86 is adjacent the interior facing surface 87 of the substrate's rear end 24. And, the central insert section 86 is between upper and lower interior facing surfaces 84 and 85 of the lateral sidewall 40 or 41 between the leading insert end 81 and rear insert end 86.

The leading insert end 81, rear insert end 86 and central insert section 83 are configured such that they define an insert component 50 or 51 that has a configuration or shape that is complimentary to a corresponding implant substrate cavity 30 or 31, as shown in FIGS. 2 and 5C. The leading insert end 81 includes a substantially flat engaging surface 81A comprising the insert component channels 52 or 53 which will engage a corresponding leading end securing tenon 21 or 23 when the insert 50 or 51 is inserted into the implant insert cavity 30 or 31. The leading insert end 81 end will also have a thickness between exterior and interior leading insert end surfaces 81B and 81C. In this embodiment, the leading insert end's 81 thickness between its interior and exterior surfaces 81B and 81C is such that the leading insert end extends into the insert cavity 30 or 31 to about a point substantially midway along the interior facing surface 82 of the substrate's leading end 22. Those of skill in the art will recognize that the leading insert end's 81 thickness between interior or exterior leading insert end 81B and 81C may vary as may be needed or desired by a physician, procedure or medical application. For example, FIGS. 6A-6B show an insert component 150 or 151 with a thickness that is the same as the lateral side walls 240 or 241, and FIGS. 7A-7C show an insert component 250 with a thickness that extends from the first lateral sidewall 240 to the second lateral sidewall 241.

The opposing rear insert end 86 includes a substantially flat engaging surface 86A which comprises insert component channels 55 and 57 which will engage corresponding rear end securing tenons 25 and 27 when the insert 50 or 51 is inserted into the implant insert cavity 30 or 31. The rear insert end 86 will also have a thickness between exterior and interior leading rear end surfaces 86B and 86C. In this embodiment, the rear insert end's 86 thickness between its interior and exterior surfaces 86B and 86C is such that the rear insert end 86 extends into the insert cavity 30 or 31 to about a point substantially midway along the interior facing surface 87 of the substrate's rear end 24. Those of skill in the art will recognize that the rear insert end's 86 thickness between interior or exterior leading insert end 86B and 86C may vary as may be needed or desired by a physician, procedure or medical application. For example, FIGS. 6A-6B show an insert component 150 or 151 with a thickness that is the same as the lateral side walls 140 or 141, and FIG. 7A shows an insert component 250 with a thickness that extends from the first lateral sidewall 240 to the second lateral sidewall 241.

The central insert section 83 will have a thickness between exterior and interior central insert section surfaces 83B and 83C. In this preferred aspect, the central insert section surface 83C, the leading insert end interior surface 81C and the rear insert end interior surface 86C together form a continuous interior insert surface for the insert components 50 and 51 which will define the implant substrate aperture 62. The central insert section 83 also includes an insert aperture 83A that extends between and through the exterior and interior central insert section surfaces 83B and 83C. The insert aperture 83A is a cylindrical aperture but may have other configurations as may be needed or desired by a physician, procedure or medical application. In this embodiment, the central insert section's 83 thickness between its interior and exterior surfaces 83B and 83C is substantially the same as the thickness of the lateral side walls 40 or 41. Those of skill in the art will recognize that the central insert sections' 83 thickness between interior or exterior leading insert end 83B and 83C may vary as may be needed or desired by a physician, procedure or medical application. For example, FIGS. 7A-7C show a single insert component 250 or 251 with a thickness that extends laterally across the substrate 220 from the first lateral sidewall 240 to the second lateral sidewall 241.

In the preferred aspect, shown in FIGS. 2-5C, there are two identical insert components 50 and 51 which respectively insert into the insert cavities 30 and 31 in opposing configurations and position on respective first and second lateral sidewalls 40 and 41. Those of skill in the art will recognize that non-identical insert components 50 and 51 may also be used as may be selected or required by a physician, procedure or medical application. In a case where insert components 50 and 51 with non-identical size or configuration are used, corresponding insert cavities 30 and 31 will have complimentary size and configuration to match that of the insert components 50 and 51 that will be positioned therein.

FIGS. 6A and 6B show a second aspect of the present disclosure. FIG. 6A is a perspective view of the spinal implant 110 according to a second embodiment of the present disclosure. FIG. 6B is a perspective view of the spinal implant 110 of FIG. 6A along section line D-D. This second embodiment is similar to that discussed above with respect to FIGS. 2-5C with the difference that the first and second insert components 150 and 151 smaller in size and this take up less volume within the implant substrate 120 when they are inserted therein. In this aspect, the first and second insert components 150 and 151 have a substantially rectangular configuration as compared to the first and second insert components 50 and 51 shown and discussed in relation to FIGS. 2-5C. In this second aspect, the implant 110 comprises an implant body or substrate 120, insert cavities 130 and 131 and complimentary first and second insert components 150 and 151 which are laterally attached to the implant body 120 at the insert cavities 130 and 131.

The implant body or substrate 120 comprises a leading end 122, a rear end 124, and first and second lateral section sidewalls 140 and 141 between the leading end 122 and a rear end 124. The first insert cavity 130 is defined and positioned between the upper and lower portions of the first lateral sidewall 140 and the substrate's leading end 122 and rear end 124. The first cavity 130 has a configuration that is complimentary to the first insert component 150 which permits the first cavity 130 to accept the complimentarily configured insert component 150 therein. An opposing second insert cavity 131 is defined and positioned between the upper and lower portions of the second lateral sidewall 141 and the substrate's leading end 122 and rear end 124. The second insert cavity 131 has a configuration that is complimentary to the second insert component 151 which permits the second cavity 131 to accept the complimentarily configured insert component 151 therein.

FIGS. 7A-7C show a third aspect of the present disclosure. FIG. 7A is a perspective view of a spinal implant 210 according to a third embodiment of the present disclosure. FIGS. 7B and 7C are perspective views of an implant substrate 220 and implant insert 250 of the implant of FIG. 7A. In this aspect, the implant 210 comprises an implant body or substrate 220, insert cavities 230 and 231 and a complimentary single insert component 250 which can be laterally attached or inserted into the implant substrate 220 at the insert cavities 230 or 231. The implant body or substrate 220 comprises a leading end 222, a rear end 224, and first and second lateral section sidewalls 240 and 241 between the leading end 222 and a rear end 224. FIGS. 7A-7C show the use of a single insert component 250. In this third embodiment, the single insert components 250 has an overall larger size and thus take up more volume within the implant substrate 220 when it is inserted therein, as compared to the first and second embodiments of FIGS. 2-6B. In the third embodiment 210, the single implant insert 250 functions similarly to the dual insert components 50, 51, 150 and 152 of the first and second embodiments 10 and 110 in the sense that the single insert component 250 is laterally inserted into complimentary configured insert cavities 230 and 231. However, in this third aspect, the there is only a single large size insert component 250 compared to or contrasted with the dual and opposing smaller first and second insert components 50, 51, 150 and 151 which take up less volume within the implant substrate 20 or 120 when they are inserted therein. In this third aspect 210, the insert component 250 is single or unitary solid body 255 with a substantially vertical central insert aperture 262 and a pair of substantially horizontal cylindrical apertures 283.

The first insert cavity 230 is defined and positioned between the upper and lower portions of the first lateral sidewall 240 and the substrate's leading end 222 and rear end 224. An opposing second insert cavity 231 is defined and positioned between the upper and lower portions of the second lateral sidewall 241 and the substrate's leading end 222 and rear end 224. The first and second cavities 230 and 231 will have configurations that are complimentary to the single insert component 250 size and configuration which thereby permits the insert cavity 230 or 231 to accept the complimentarily configured insert component 250 therein. In this aspect, the insert component 250 is a single body 255 that will be inserted and positioned in the implant substrate 220 and which will laterally span across the implant 210 and the insert cavities 230 and 231 between the first and second lateral side walls 240 and 241, as shown in FIG. 7A. The single implant component 250 further includes a central insert aperture 262 which will align with corresponding upper and lower substrate apertures 263 when the single insert component 250 is positioned and coupled to the substrate 220. The central insert aperture 262 and upper and lower substrate apertures 263 permit the insertion of a graft material which assists in promoting fusion of the adjacent vertebrae at the disc space 101 where the implant 210 is inserted therein.

In a preferred spinal implant embodiment 10, 110 or 210, the implant substrate 20, 120 and 220 is preferably a titanium (Ti) material which is radiopaque and osseoconductive biomaterial, and the opposing insert components 50, 51, 150, 151 and 250 are preferably polyetheretherketone (PEEK) polymer materials which are radiolucent biomaterials with a lower modulus of elasticity. As discussed above, the implant substrate's 20, 120 and 220 titanium serrations or teeth provide an exterior bone-contacting surface which enables immediate and strong fixation of the implant 10, 110 and 210 to adjacent vertebrae in the disc space. The implant substrate's 20, 120 and 220 titanium serrations or teeth also assist in long-term osseointegration for the device while allowing for radiographic assessment of osseointegration between endplates 103 and 107 and implant's 10, 110 and 210 surfaces. The radiolucent PEEK insert components 50, 51, 150, 151 and 250 allows for radiographic assessment of fusion or bridging bone mass across the disc space while reducing stress-shielding effects. These are advantageous characteristics and properties that are contemplated for the implant embodiments 10, 110, 210 disclosed herein which couple an osseoconductive radiopaque implant substrate 20, 120 and 220 with one or more radiolucent insert components 50, 51, 150, 151 and 250.

In a preferred aspect, where the implant substrate 20, 120 and 220 is titanium and the insert components 50, 51, 150, 151 and 250are PEEK material, the titanium implant substrate 20, 120 and 220, which is a single piece or component, will carry the load or take on compressive force that is exerted on the implant 10, 110 and 210 in the disc space 101. Further, due to the configuration and positioning of the PEEK insert components 50, 51, 150, 151 & 250 inside the substrate insert cavities 30, 31, 130, 131, 230 and 231, the PEEK insert components 50, 51, 150, 151 and 250 will act as a support strut and take or and support compressive load between the implant 10, 110 and 210 upper and lower substrate surfaces. This aspect leads to an advantageous characteristic that there is load sharing between the between titanium implant substrate 20, 120 and 220 and PEEK insert components 50, 51, 150, 151 and 250.

The embodiments of FIGS. 2-7C disclose spinal implants 10, 110 and 210 with different sized implant substrates 20, 120 and 220, and insert cavities 30, 31, 130, 131, 230 and 231 that are configured to laterally receive and lockingly engage complimentarily configured insert components 50, 51, 150, 151 and 250. As such, the volume taken up in the substrate insert cavities 30, 31, 130, 131, 230 and 231 by the one or more insert components 50, 51, 150, 151 and 250 will vary depending on which embodiment is used. Or alternatively, the ratio of material between the implant substrate 20, 120 and 220 and insert components 50, 51, 150, 151 and 250 will vary depending on which embodiment is used to make up the coupled spinal implant embodiment 10, 110 and 210. Those of skill in the art will readily recognize that the volume of the implant substrate 20, 120 and 220 taken up by the one or more insert components 50, 51, 150, 151 and 250, or the ratio of material between the implant substrate 20, 120 and 220 and insert components 50, 51, 150, 151 and 250 can be selectively varied or controlled for a particular spinal implant as may be selected or required by a surgeon or medical procedure or application.

In the disclosed embodiments, the volume of interior implant substrate 220 taken up by the one or more insert components 50, 51, 150, 151 and 250 will vary depending on spinal implant 10, 110 or 210 used. As shown in FIGS. 6A-6B, the second embodiment 110 has first and second insert components 150 and 151 which are smaller in size than the first and second insert components 50 and 51 of the preferred implant embodiment 10, and therefore take up less volume within the implant substrate 120 when they are inserted therein. This is the case since, the first and second insert components 150 and 151 are smaller in size than the first and second insert components 50 and 51, shown FIGS. 2-5C. In the third implant embodiment 210, shown in FIGS. 7A-7C, the third embodiment 210 has a single insert component 250 which is larger in size than either the insert components 50, 51, 150 and 151 of the preferred or second implant embodiments 10 and 110, respectively. Therefore, the single insert component 250 will take up more volume within the implant substrate 220 when it is inserted therein. This is the case since the single insert component 250 is larger in size than either of the first and second insert components 50, 51, 150 and 151 of the preferred and second implant embodiments 10 and 110, respectively. Thus, as the size of the insert component 50, 51, 150, 151 and 250, and correspondingly the substrate insert cavities 30, 31, 130, 131, 230, and 231 increases from smaller (second embodiment 120) to larger (third embodiment 220) the implant substrate's 20, 120 and 220 corresponding material mass or volume will decrease from larger (third embodiment 220) to smaller (second embodiment 120).

In the preferred aspect, where the implant substrate 20, 120 and 220 is a Ti material and the insert component 50, 51, 150, 151 and 250 is a PEEK polymer material, as the size, mass or volume of the insert component 50, 51, 150, 151 and 250 increases from smaller (second embodiment 120) to larger (third embodiment 220) there results a larger mass or volume of PEEK insert component material 50, 51, 150, 151 and 250 compared to the Ti implant substrate 20, 120 and 220 mass or volume which is decreasing as the mass of PEEK insert component 50, 51, 150, 151 and 250 increases, where the exterior size of the implant substrate does not change. Thus, the ratio of the materials making up the implant 10, 110 and 210 would be more PEEK insert component polymer material 50, 51, 150, 151 and 250 than Ti implant substrate material 20, 120 and 220. This aspect results in an advantageous implant characteristic such that, from an imaging standpoint, the spinal implant 10, 110 and 210 would have more radiolucent insert component material 50, 51, 150, 151 and 250 and thus greater resultant spinal implant 10, 110 and 210 radiolucent properties. Additionally, when there is more PEEK insert component material 50, 51, 150, 151 and 250, the spinal implant's 10, 110 and 210 overall compressive modulus or stiffness is less as compared to when there is more titanium implant substrate material 20, 120 and 220. Lower overall compressive modulus/stiffness for the spinal implant 10, 110 and 210 is an advantageous aspect since such an implant 10, 110 and 210 more closely approximates the modulus of the bone. Those of skill in the art will thus recognize that the size of the PEEK insert component 50, 51, 150, 151 and 250 can be increase to improve overall implant 10, 110 and 210 compressive stiffness, such that one could transition from less PEEK insert mass (the smaller two insert piece second embodiment 120) to a single large PEEK insert mass (the larger single insert piece third embodiment 220) which would then have better overall implant compressive stiffness characteristic and radiolucent properties. The size, mass and volume of the PEEK insert component 50, 51, 150, 151 and 250 and corresponding complimentary implant substrate 20, 120 and 220 size, mass and volume can be selected for a particular spinal implant as may be desired or required by a surgeon or medical procedure or application.

In the disclosed embodiments of FIGS. 2-7A, a spinal implant, commercialized by Medtronic, Inc, under the trademark CLYDESDALE®, is contemplated as using and embodying the advantageous aspects of the spinal implant 10, 110 and 210 disclosed herein. Those of skill in the art will readily recognize that other implant sizes and configuration designs may use or incorporate the advantageous aspects of the spinal implant 10, 110, and 210 disclosed herein. This includes implants having different leading end and rear end configurations. For example, the unique and advantageous aspects of the spinal implant 10, 110 and 210 disclosed herein may be implemented and used in others spinal implants commercialized by a third party, including spinal implants commercialized by Medtronic, Inc, under the trademarks CAPSTONE®, CRESCENT®, etc., along with associated or corresponding delivery and insertion implant instruments. Those of skill in the art will further recognize that implant 10, 110 and 210 could also comprise a substrate 20, 120 and 220 and insert components 50, 51, 150, 151 and 250 with resultant implant walls which are angled relative to one another so as to achieve a desired or selected kyphosis, lordosis, or lateral wedge effect when inserted in the disc space 101. In other embodiments, an implant wall or surface may extend obliquely from an adjacent wall rather than orthogonally. Also, an implant's walls could be tapered, sloped, angled, or curved, including convex, bi-convex and concave curving, depending on a particular medical application need or requirement.

The spinal implants 10, 110 and 210 disclosed in this disclosure are preferably comprised of a biocompatible osseoconductive radiopaque implant substrate with one or more attachable or insertable biocompatible radiolucent insert components which are configured and adapted for insertion into and positioning in an intervertebral disc space so as to contact against adjacent vertebral members. The implant substrate 20, 120 and 220 includes serrations or teeth 70, 170 and 270 which provide or promote fixation as well as long-term osseointegration for the implant device 10, 110 and 210 while allowing assessment of osseointegration between vertebral endplates and spinal implant surfaces. Fusion and osseointegration can be improved and accelerated through the use and application of a Hydroxyapatite or HA coating on the spinal implant surfaces and substrate serrations or teeth 70, 170 and 270. The biocompatible insert component 50, 51, 150, 151 and 250 is preferably a polyetheretherketone (PEEK) polymer material. The spinal implant 10 and 200 contemplated herein allows radiographic assessment of fusion and the bridging bone mass across the disc space while reducing stress-shielding effects.

The biocompatible insert component 50, 51, 150, 151 and 250 is preferably a radiolucent biocompatible materials such as PEEK and carbon fiber reinforced PEEK, etc., however, those of skill in the art will recognize that other insert component material may also be used, including among others, polymer material, homopolymers, co-polymers and oligomers of polyhydroxy acids, polyesters, polyorthoesters, polyanhydrides, polydioxanone, polydioxanediones, polyesteramides, polyaminoacids, polyamides, polycarbonates, polylactide, polyglycolide, tyrosine-derived polycarbonate, polyanhydride, polyorthoester, polyphosphazene, polyethylene, polyester, polyvinyl alcohol, polyacrylonitrile, polyamide, polytetrafluorethylene, poly-paraphenylene terephthalamide, polyetherketoneketone (PEKK); polyaryletherketones (PAEK), cellulose, carbon fiber reinforced composite, and mixtures thereof. The insert component 50, 51, 150, 151 or 250 may also be a resorbable polymer material. The resorbable polymer, includes, but not limited to, polylactide, polyglycolide, copolymers of polylactide and polyglycolide, polycaprolactone, polyorthoester, tyrosine-polycarbonate, polurethane, etc. In the case where the insert component 50, 51, 150, 151 and 250 is a restorable material, the insert components 50, 51, 150, 151 and 250 is resorbed over a period of time such that the restorable insert component 50, 51, 150, 151 and 250 is gradually resorbed away as fusion within the disc space 101 progresses and the resorbable insert components 50, 51, 150, 151 and 250 is not needed anymore. As the resorbable insert component 50, 51, 150, 151 and 250 is resorbed there is left a void or cavity where the insert component 50, 51, 150, 151 and 250 existed before it was resorbed, and bone can grow into the void or cavity.

The biocompatible radiopaque and osseoconductive implant substrate 20, 120 and 220 is preferably a Titanium (Ti) or metallic material. However, those of skill in the art will recognize that other metallic materials may also be used, including, among others, stainless steel, titanium alloys, nitinol, platinum, tungsten, silver, palladium, gold, cobalt chrome alloys, shape memory nitinol and mixtures thereof. Additionally, the metallic material may have a porosity aspect in order to improve fixation of the implant. The bone-contacting surfaces, serrations or teeth of the implant substrate's metallic material may have porosity of appropriate or desired sizes and geometry or configuration for optimal and rapid bony in growth. The bone-contacting surfaces, serrations or teeth porosity may have pores that are non-connected or interconnected pores with pore size diameter in the range between 1 to 1000 micrometers, preferably between 50 and 250 micrometers. The porosity may have predetermined patterns or have a porosity that has a random geometry or configuration in nature. The porosity can be further coated or filled with osseoconductive and/or osseoinductive biomaterials such as hydroxyapatite (HA) and human recombinant bone morphogenic protein (rh BMP2). Those of skill in the art will recognize that the pore sizes, pore configuration, pore coating, and/or pore inter-connectivity aspect may be selected or vary for a particular spinal implant 10, 110 or 210 depending on needs or requirements of a physician, procedure or medical application. Further, the biocompatible substrate 20, 120 and 200 and insert components 50, 51, 150, 151 and 250 used may depend on the patient's need and physician requirements. The spinal implant substrate 20, 120 and 220 and insert components 50, 51, 150, 151 and 250 can be made or manufactured by typical or known techniques and methods know to those of skill in the art, including among others, machining, molding, extrusion, stamping, laser processing, water-jet cutting or combination thereof.

The implant 10, 110 or 210 may be implanted in the disc space 101 using known methods, procedures and approaches, including a posterior (PLIF), direct lateral (DLIF), anterior (ALIF), translateral (TLIF) or any other suitable oblique direction and approach, as those of skill in the art will recognize. Further, a spinal implant may be delivered and inserted through known surgical technique and procedures, including: open, mini-open, minimal access spinal technologies (MAST) or other minimally invasive surgical (MIS) techniques.

In one approach, the implant 10, 110 or 210 is inserted via a posterior (PLIF) approach, for example as shown in FIG. 1. In one aspect, the implant 10, 110 or 210, shown in FIGS. 2, 6A and 7A, will have a selected or desired physical shape and size for use in a spinal medical procedure. Those of skill in the art will readily recognize that the implant 10, 110 or 210 may take on any shaped desired or required for a particular medical use or application. Further, those of skill in the art will recognize that the implant can also be a dynamic vertebral implant device, with varying shape and size depending on the medical application where the implant used.

Prior to insertion, known medical instruments and tools may be used to prepare the intervertebral disc space 101, including pituitary rongeurs and curettes for reaching the nucleus pulposus or other area in the disc space 101. The disc space 101 may be prepared with a partial or complete discectomy. Ring curettes may be used as necessary to scrape abrasions from the vertebral endplates 103 and 107. Using such instruments, a location which will accept the implant 10, 110 or 210 is prepared in the disc space 101. Those of skill in the art will recognize that the implant 10, 110 or 210 may be positioned at any desired location between the adjacent vertebral bodies 103 and 107 depending on the surgeon's need and the performed surgical procedure or medical application.

The implant is then inserted into the prepared disc space 101 using insertion instruments which are appropriate with the shape and configuration of the implant and surgical procedure to be used. A medical imaging technique and device may be used to visualize the implant 10, 110 or 210 during the insertion procedure by taking advantage of the implant's radiolucent and radiopaque properties. During the insertion step, the enhanced implant visualization will permit the surgeon to better maneuver and control the trajectory, position and orientation of the implant 10, 110 or 210 into the vertebral disc space 101 and through the surrounding patient anatomical environment.

The implant 10, 110 or 210 is then delivered into the intervertebral disc space 101 and positioned in a selected location and orientation between the end plates 103 and 107 of the adjacent vertebral bodies 100 and 105. The implant is inserted into the disc space 101 such that the implant upper and lower surface serrations or teeth 70, 170 and 270 are positioned between and adjacent to the upper and lower vertebral endplates 103 and 107. The substrate serrations 70, 170 and 270 may engage the vertebral endplates 103 and 107 to provide stability to the implant 10, 110 or 210. Once implanted, the implant's upper surface serrations or teeth 70, 170 and 270 will contact the upper vertebral end plate 103 to form an interface between the implant 10, 110 or 210 and the upper vertebral body 100. Also, the implant's lower surface serrations or teeth 70, 170 and 270 will contact the lower vertebral end plate 107 to form an interface between the implant 10, 110 or 210 and the lower vertebral body 105. After the insertion of the implant 10, 110 or 210 between the vertebral bodies 100 and 105 has been completed, the implant graft material will promote the fusion or joining together of the vertebral bodies 100 and 105.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

While embodiments of the invention have been illustrated and described in detail in the present disclosure, the disclosure is to be considered as illustrative and not restrictive in character.

While embodiments of the invention have been illustrated and described in the present disclosure, the disclosure is to be considered as illustrative and not restrictive in character. The present invention may be carried out in other specific ways than those herein set forth without departing from the scope and essential characteristics of the invention. The present embodiments are, therefore, to be considered in all respects as illustrative and not restrictive, and all changes and modifications that come within the spirit of the invention are desired to be protected and are to be considered within the scope of the disclosure. Further, all changes coming within the meaning and equivalency range of the appended claims are also intended to be embraced therein.

What is claimed:

1. A spinal implant for insertion into and positioning in an intervertebral disc space, the implant comprising:
an implant substrate comprising at least one insert cavity and bone securing serrations; and
at least one insert component;
wherein the at least one insert component is configured to be securely coupled to the implant substrate via the at least one insert cavity to thereby form the spinal implant.

2. The implant of claim 1, wherein the insert component is internally positioned inside the implant substrate when securely coupled to the implant substrate.

3. The implant of claim 1, wherein the at least one insert cavity and the at least one insert component are securely coupled via a mechanical interlock configuration.

4. The implant of claim 1, wherein the at least one insert component is securely coupled to the implant substrate via entry into a lateral sidewall insert cavity.

5. The implant of claim 1, wherein the spinal implant comprises two insert components configured to be securely coupled to the implant substrate via a corresponding insert cavity to thereby form the spinal implant.

6. The implant of claim 1, wherein the spinal implant comprises one insert component configured to be securely coupled to the implant substrate via a first or second insert cavity to thereby form the spinal implant.

7. The implant of claim 6, wherein the one insert component, when securely coupled to the implant substrate, laterally spans across the implant substrate between a first and second lateral sidewall.

8. The implant of claim 1, wherein
the implant substrate is comprised of a radiopaque material; and
the at least one insert component is comprised of a radiolucent material.

9. The implant of claim 1, wherein the at least one insert component is polyetheretherketone (PEEK) or a resorbable material.

10. The implant of claim 1, wherein the implant substrate is a metallic material.

11. The implant of claim 10, wherein the metallic material is titanium (Ti) or a titanium (Ti) alloy.

12. The implant of claim 1, wherein the implant substrate comprises a Hydroxyapatite (HA) layer.

13. A spinal implant for insertion into and positioning in an intervertebral disc space, the implant comprising:
an implant substrate comprising at least one insert cavity and bone securing serrations; and
at least one insert component configured to be positioned inside the at least one insert cavity;
wherein the at least one insert component is configured to be securely coupled to the implant substrate via a lateral sidewall entry into the at least one insert cavity to thereby form the spinal implant.

14. The implant of claim 13, wherein the at least one insert cavity and the at least one insert component are securely coupled via a mechanical interlock configuration.

15. The implant of claim 13, wherein the spinal implant comprises two insert components configured to be securely coupled to the implant substrate via a corresponding insert cavity to thereby form the spinal implant.

16. The implant of claim 13, wherein the spinal implant comprises one insert component configured to be securely coupled to the implant substrate via a first or second insert cavity to thereby form the spinal implant.

17. The implant of claim 16, wherein the one insert component, when securely coupled to the implant substrate, laterally spans across the implant substrate between a first and second lateral sidewall.

18. The implant of claim 13, wherein the at least one insert component is polyetheretherketone (PEEK) or a resorbable material.

19. The implant of claim 13, wherein the implant substrate is titanium (Ti) or a titanium (Ti) alloy.

20. The implant of claim 13, wherein the implant substrate comprises a Hydroxyapatite (HA) layer.
